(19) United States
(12) Patent Application Publication
(10) Pub. No.: US 2013/0331802 A1
(43) Pub. Date: Dec. 12, 2013

(54) Wound Treatment Medium and Method of Packaging and Use

(71) Applicants: Cory Collinge, Fort Worth, TX (US); Kevin Kelly, Fort Worth, TX (US)

(72) Inventors: Cory Collinge, Fort Worth, TX (US); Kevin Kelly, Fort Worth, TX (US)

(21) Appl. No.: 13/907,005

(22) Filed: May 31, 2013

Related U.S. Application Data
(60) Provisional application No. 61/656,085, filed on Jun. 6, 2012.

(51) Int. Cl. A61J 1/10; A61F 13/00; A61M 37/00

(52) U.S. Cl. CPC A61J 1/10; A61M 37/00; A61F 13/00008
USPC 604/306

(57) ABSTRACT

A sterile wound treatment kit includes a plastic container pouch with a sealed outer periphery. A drug absorbing medium is located within the pouch interior. A syringe accepting fitting is located on the pouch for discharging a treatment drug from a syringe into the pouch interior shortly before treating a patient. The drug absorbing medium then contacts and absorbs the treatment drug. The pouch can be opened at the time of use to allow access to the drug absorbing medium so that the medium can be applied to a wound site. The pouch can also be equipped with a dispensing nozzle for dispensing the treatment medium.

### CROSS-REFERENCE TO RELATED APPLICATION

The present Application claims priority to U.S. Provisional Patent Application Ser. No. 61/656,085 filed Jun. 6, 2012 and entitled: “Wound Treatment Medium and Method of Packaging and Use” by the same inventors.

### BACKGROUND OF THE INVENTION

1. Field of the Invention

The invention relates to a device and method for helping to prevent infection and for aiding tissue repair in the treatment of an injured mammal that involves the medical treatment and healing process of injured soft tissue and damaged osseous material such as is found in the case of a compound fracture wound.

2. Description of the Prior Art

Wound management is a significant portion of nearly all modern medical practice environments. Wounds occur from a large variety of sources including, to name a few, burns, blunt trauma, chronic ulceration, skin lacerations, tissue abscesses and irritation, open bone fractures (compound fracture), and pressure sores. Such wounds and their treatment constitute a large percentage of the treatment provided to medical patients. The best treatment for these wounds is complete a cleansing and sterile treatment, as close to the time and site of injury as possible.

Compound or “open” fractures are a relatively common occurrence in society today. In a compound fracture the bone undergoes sufficient trauma that it literally breaks into pieces and tears through the skin. Once the bone is in communication with the outer environment, the wound and fracture site is then highly susceptible to infection. In order to properly heal, the bone must be placed back into alignment and held in place for a sufficient length of time for the piece of bone to be fused together by way of new bone growth. The bone must be properly aligned or “reduced” for the bone to heal properly. In order to hold the two pieces of bone in the correct position, surgical intervention is usually necessary, typically involving the placement of a suitable retention device. The retention device can be a plate or apparatus which is attached by screws or some similar mechanism on the outside of the bone. In the alternative, a retention pin or rod which is disposed in the cavity extending through the center of the bone is used in some cases.

Osteomyelitis is a bone infection caused by destructive microorganisms, most commonly, staphylococcus aureus. Very often, infection reaches the bone via compound fracture. It is known that a large number of open fractures are contaminated with various types of bacterial organisms prior to any surgical intervention. The growth of microorganisms in an open fracture environment is enhanced by the impaired vascularity, debris deposited in the wound, and loss of skeletal stability. Some of the worst and most difficult to treat infections are “hospital-acquired” or “nosocomial” infections, where virulent hospital flora may cause the infection. This has clearly been seen for cases of traumatic wounds and compound fractures where the wounds have been left without aseptic wound coverage in the hospital and have become infected. Infection may seriously complicate the healing process and may lead to extended hospital stays, loss of limb, and in some cases, even death. Infections are generally recognized as being a primary cause of non-union and bony instability following open fractures. Thus, a chief objective in the treatment of open fractures is not only to stabilize osseous structures, but also to prevent soft tissue and bony infections.

There is often an 8-24 hour window for surgical treatment of wounds such as the compound fractures described above. This may be due to the lack of immediate availability of a surgeon, to the lack of availability of needed hospital facilities, and for a variety of other causes. As a result, it is often necessary to treat wounds of the above type with some sort of wound cleansing and debridement technique as a temporary treatment measure until surgery can be performed.

A large number of methods for wound cleansing and debridement have been developed in the past. Those methods include wound cleansers such as povidone-iodine, hydrogen-peroxide, acetic acid, and chlorinated solutions, which however, have a toxic effect on cells. Other types of wound cleaning and debridement include the use of broad spectrum antibiotics, piston type syringe irrigation, wet to dry saline gauze dressings, surgical/mechanical debridement, enzymatic debridement, antibiotic impregnated beads, and pulsed lavage. The utilization of antibiotic therapy has been shown to be extremely important, and perhaps the most important, intervention in reducing the incidence of infection in the case of compound fracture wounds. A device or medium that can deliver a high local dose of antibiotic is thus desirable. The prognosis of patients undergoing antimicrobial therapy is determined by the bactericidal level of antibiotics delivered at the locale of the infected site.

A persistent problem with treating any localized infection by systemic administration of antibiotics is that the relationship between the assayed serum antibiotic concentration and the level present at the site of the infection is inconsistent, especially when the local site is traumatized tissue. Antibiotic concentrations are often subtherapeutic due to impaired vascularity at the fracture site, devitalized bony fragments, and/or associated systemic complications. Consequently, high doses of parenteral antibiotics must often be used to achieve adequate local concentrations. The high doses are not only costly, but more importantly increase the incidence of systemic side effects. The treatment of infection due to compound fracture, or other cause, has been known to fail due to the inability to achieve adequate antibiotic levels at the infected local.

Traumatic soft tissue wounds are also a setting in which antibiotics must be employed in a timely manner. Frequent admissions to the Emergency Department are the result of blunt or penetrating trauma and rarely gun shot wounds. Complicating the care of these patients, these wound are often contaminated by skin and environmental bacteria. Direct administration of antibiotics to the wound may help reduce the risk of local and systemic infections.

Chronic soft tissue wounds such as decubitus ulcers, diabetic wounds, and others present a great challenge to the treatment teams. These areas of devitalized tissue require frequent dressing changes and the use of wound cleansers that can have toxic effects on the healing tissues. Further complicating the management of these patient's wounds are the precipitating cause of the infection. Patients who suffer from decubitus ulcers are often bed bound which can falsely decrease serum creatinine concentrations which are used as a marker of renal function. Inaccurate serum creatinine levels due to decreased muscle mass and lack of mobility mask serious renal dysfunction. Systemic antibiotic dosing can be difficult without an accurate assessment of renal function and may lead to overdosing of antibiotics that can have serious toxic side effects.

A need, therefore, has previously been recognized for an improved antibiotic treatment medium and method which would be appropriate for use with acute and chronic soft tissue wounds and which would address the above discussed shortcomings of the prior art.

Applicant's own issued U.S. Pat. No. 7,435,423, issued Oct. 14, 2008, entitled “Wound Treatment Medium and Method of Packaging and Use”, by the same inventors, describes an improved treatment medium and method for addressing many of the shortcomings of the prior art. Despite the advantages offered by the device and method described in that issued patent, additional features have been developed in recent years which add additional capabilities and offer further advantages over the prior art.

### SUMMARY OF THE INVENTION

Shown herein are a wound treatment kit and method of use which are appropriate for use in applying a wound treatment medium onto an injured portion of a mammalian body, the treatment medium being impregnated with a treatment drug, whereby the drug is released from the medium to contact and penetrate the wound area. The wound area may particularly relate to compromised bone, for example, the type found in compound fracture wounds.

The wound treatment kit of the invention includes a container pouch formed of flexible plastic, the container pouch having an exterior, an interior and a sealed outer periphery, the sealed outer periphery forming a sterile interior region which isolates the pouch interior from a surrounding environment. A drug absorbing medium is located within the pouch interior and initially isolated from the surrounding environment by the sealed periphery. A syringe accepting fitting, such as a Leur Lock or other fitting, is located on the pouch for discharging a drug treatment from a syringe into the pouch interior. The pouch interior is selectively sized relative to the drug absorbing medium to allow the medium to be shaken or manipulated within the pouch interior once a drug is injected into the sterile interior region, whereby the drug absorbing medium will contact and evenly absorb the treatment drug. The preferred treatment drugs are broad spectrum antibiotics.

The container pouch can conveniently be formed of two overlaying sheets of flexible plastic, the sheets of flexible plastic having an adhesive applied around an outer periphery thereof to form a sealed pouch. The Leur Lock or other fitting has a syringe receiving end and an opposite end. The opposite end can be engaged between the two overlaying sheets of flexible plastic at a point on the periphery thereof, the opposite end also extending at least part way into the pouch interior region for dispensing a treatment drug into the interior region. In one form, the container pouch can be peeled one sheet from the other in a “peel pack” form or the flexible plastic used to form the container pouch has a thickness which is selected to allow the pouch to be opened by tearing by hand or to be easily cut with a pair of scissors.

In one form of the invention, the drug a absorbing medium is a malleable biomaterial instead of a sponge. In that case, the pouch can also be equipped with a dispensing nozzle having a nozzle cap for initially closing the nozzle, or can be or the twist on-off design. The dispensing nozzle communicates the interior and exterior of the pouch, whereby once a drug is injected into the sterile interior region and contacts and impregnates the biomaterial absorbing medium, the biomaterial absorbing medium can be discharged through the nozzle from the pouch interior onto a wound site. The nozzle would be useful for dispensing biomaterial treatment mediums which include, for example, Type I collagen in either dry powder form or semi-wet form that can be supersaturated with a selected antibiotic. Another treatment medium that could be dispensed through the nozzle would be Keratin Hydrogel in either powder form or semi-wet form that can be supersaturated with a selected antibiotic. Another choice of drug absorbing medium is Chitosan in either dry powder that can be congealed or in the form of a sponge that can be supersaturated with a selected antibiotic. Where the sponge is present, the pouch would be torn apart or cut to dispense the treatment medium.

In the method of the invention, a wound such as a compound fracture wound or other as described, is treated by providing a sponge within the sterile interior region of a container pouch as previously described., or by using Type I collagen, Keratin Hydrogel or Chitosan, as previously described. The pouch can be sold in bulk to medical treatment facilities. At the facility, the appropriate medical personnel decide which treatment drug is appropriate for the wound in question and the drug is then injected into the container pouch by dispensing the drug from a syringe through the Leur Lock or other fitting or region on the pouch. The drug is evenly dispersed through the drug absorbing medium by shaking or massaging the container pouch for several seconds. The container pouch is then opened, either by peeling the plastic apart from one another, tearing by hand, or by cutting the pouch with scissors, thereby allowing access to the treatment medium. Alternatively, where the pouch is equipped with a dispensing nozzle, the nozzle can be opened and the treatment sprayed on the wound site by squeezing the pouch. Any of a number of different drugs of choice may be selected by a physician for injection into the pouch interior for ultimate application to the wound.

The systems of the invention are able to localize the concentration of antibiotic to the area of injury, for example, the bone and surrounding local traumatized tissues of an open fracture, or at the site of a chronic wound such as a decubitus ulcer, while avoiding problems associated with administering high doses of antibiotic systemically.

The medium and delivery system of the invention also incorporates simple and inexpensive sterile packaging, is simple in design and utilizes variations of existing commercially available components of medical supplies of the type traditionally used in wound treatment. Such medium and delivery systems could be utilized by the military in the field of battle as well as during man made and natural disasters where the infrastructure is not sufficient to support parenteral antibiotic administration.

Additional objects, features and advantages will be apparent in the written description which follows.

### BRIEF DESCRIPTION OF THE DRAWINGS

FIG. 1 is a perspective view, partly broken away, of one of Applicant's prior art wound treatment kits prior to impregnating the treatment medium with a treatment drug.

FIG. 2 is a simplified view of a compound fracture wound of the type which might be treated with one of Applicant's would treatment kits.

FIGS. 3-5 are simplified illustrations of the steps involved in utilizing one of the wound treatment kits of FIG. 1 in treating a wound.

FIG. 6 is a view similar to FIG. 1, but showing an improved wound treatment kit of the invention.

FIG. 7 is a close up view of a portion of a wound treatment kit of the invention showing another type of fitting for the container pouch.

FIG. 8 is another close up view of a pouch having a butyl patch region.

FIG. 9 is a simplified view of the use of the wound treatment kit of the invention, showing the pouch being squeezed to dispense a treatment to a wound area.

### DETAILED DESCRIPTION OF THE INVENTION

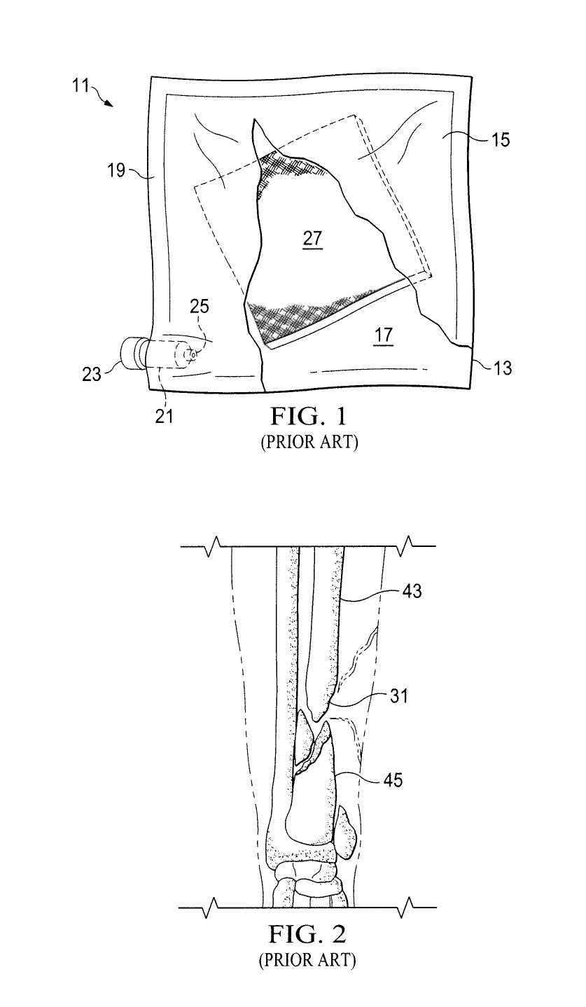

Applicant's previously issued U.S. Pat. No. 7,435,423, as has been mentioned, showed a wound treatment medium and method of packaging and use which was an improvement over the prior art. It is perhaps easiest to understand the further improvements which the present invention offers by first describing the prior art system. Turning to FIG. 1, there is shown one of Applicant's sterile wound treatment kits, as described in U.S. Pat. No. 7,435,423, the kit being designated generally as 11. The kit 11 comprises a container pouch formed of a flexible material, such as a suitable plastic. The container pouch 13 has an exterior 15, an interior 17 and a sealed outer periphery 19. The sealed outer periphery 19 forms a sterile interior region 17 which isolates the pouch interior from a surrounding environment. The flexible plastic chosen for the container pouch can be any convenient commercially available material such as the commonly known polyolefins such as polypropylene, polyethylene, etc.

Preferably, the container pouch 13 is formed of two overlaying sheets of flexible plastic. The sheets of flexible plastic having an adhesive applied around the outer periphery 19 thereof to form the sealed pouch. Alternative sealing techniques can also be utilized, such as by forming the sealed peripheral region by heat treating and thereby sealing the peripheral edges of the pouch.

A syringe accepting fitting, such as the Leur-Lock or other fitting 21 is located on the pouch for discharging a treatment drug from a syringe into the pouch interior 17. The syringe accepting fitting 21 has as syringe receiving end 23 and an opposite end 25. The opposite end 25 can be engaged between the two overlaying sheets of flexible plastic at a point on the periphery thereof. The opposite end 25 extends at least part way into the pouch interior 17, or otherwise communicates with the pouch interior, for dispensing a treatment drug into the interior region 17.

The flexible plastic material which is selected for the container pouch 13 preferably has a thickness and strength properties which allow the pouch to be opened by simply tearing the pouch by hand. Alternatively, a thicker or stronger material may be utilized and the pouch may be opened by cutting with scissors or otherwise puncturing the pouch.

A drug absorbing medium 27 is located within the pouch interior 17 and initially isolated from the surrounding environment by the sealed periphery 19. The prior art drug absorbing medium is a piece of sterile gauze pad, although other porous carrier mediums may be employed as well. The dimensions of the pouch interior 17 are selectively sized relative to the size of the drug absorbing medium 27 to allow the medium to be shaken within the pouch interior 17 once a drug is injected to the sterile interior region through the syringe accepting fitting 21. In this way, the drug absorbing medium 27 will contact and evenly absorb the treatment drug. The particular treatment drug selected will vary depending upon the nature and extent of the wound and other factors. Preferably, the treatment drug is a broad spectrum antibiotic. Known antibiotics of this general type include, e.g., Cephazolin, Tobramycin and Gentamycin. Other antibiotic drugs may also be used, including those having specialized purposes, such as those antibiotics which are effective under anaerobic conditions.

The prior art method of treating a wound by applying a sterile treatment medium will now be explained with reference primarily to FIGS. 3-5. A container pouch 29 (FIG. 3) is provided formed of a flexible plastic, as previously described. The sterile drug absorbing medium, in this case a sterile gauze pad 31, is placed within the pouch interior with the sealed outer periphery 31 forming a sterile interior region which isolates the pouch interior from the surrounding environment.

A treatment drug of choice is then discharged into the pouch interior by installing the needle of the syringe 35 within the barrel of the syringe accepting fitting 37 and discharging the drug from the syringe into the pouch interior. The pouch is then agitated in some fashion, as by shaking by hand, in order to evenly impregnate the gauze pad 31 with the treatment drug.

Typically, the sealed pouch 29 is supplied in bulk to the medical facility and has an extended shelf storage life. The treatment drug would not be typically discharged through the syringe accepting fitting 37 until shortly before the time of expected use. This would insure that the treatment drug was relatively fresh and effective.

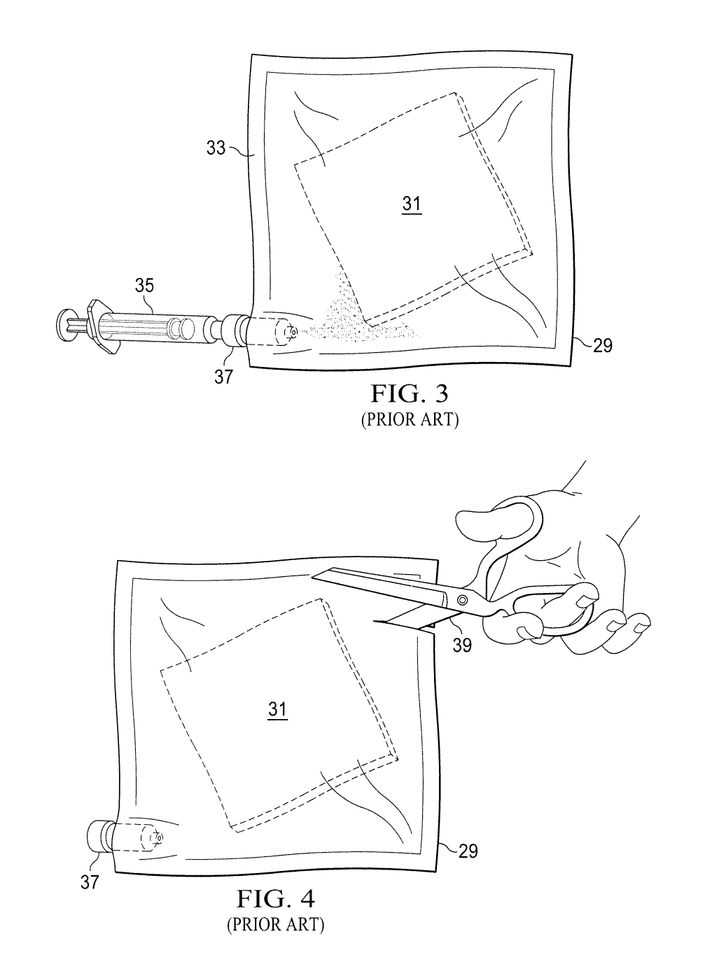

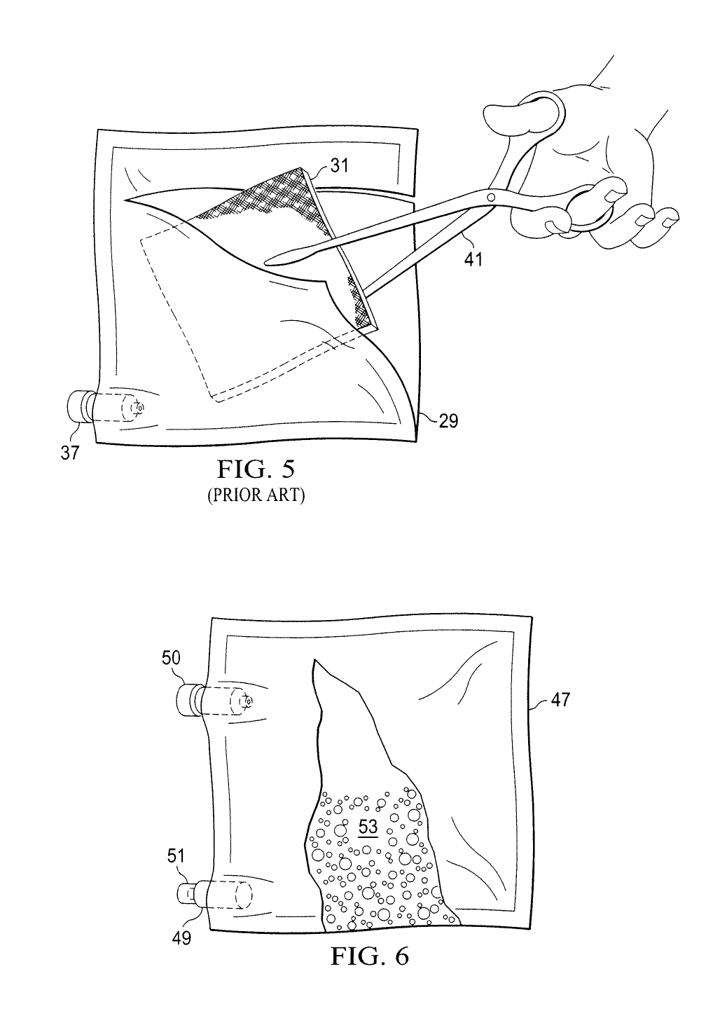

After the treatment medium has had sufficient time to absorb the treatment drug, the container pouch 29 would be opened in some fashion and the sterile gauze pad 31 applied to the wound site. In one embodiment of the invention, the plastic material of the container pouch 29 is peeled one side away from the other to maintain maximal sterility. A sufficiently thin or weak is also envisioned to allow the pouch to be opened by simply tearing by hand. In some cases, it may be necessary to puncture the pouch in some other fashion, as shown by cutting with scissors 39 in FIG. 4. FIG. 5 shows the sterile gauze pad 31 which is now impregnated with the treatment drug being grasped with a sterile instrument 41 which is used to apply the gauze pad to the wound site.

FIG. 2 is a simplified view of a compound fracture wound of the leg in which one of the large bones is actually split into two separate pieces 43, 45. A gauze pad 31 has been placed over the bone ends 43, 45 at the wound site and the wound has been temporarily covered. This technique might be used to temporarily stabilize a wound overnight until the necessary surgery to fix the severed bones and address the soft tissues could be accomplished, for example, the next morning. These techniques could also be employed, as described above, in the treatment of penetrating wounds, as well as chronic wounds that are the result of diabetes, venous stasis ulcers, decubitus and other soft tissue ulcerations.

The improvements toward which the present invention are directed will now be described with respect primarily to FIGS. 6-9 of the drawings. As shown in FIG. 9, the pouch 47 is equipped with a dispensing nozzle 49, having a nozzle cap 51. Alternatively, the nozzle might be of the twist-open variety. In the case of the pouch of FIG. 6, instead of a sterile gauze pad, the drug absorbing medium 53 can be a naturally occurring or synthetically derived biocompatible material or delivery medium based upon or incorporating such a biocompatible material which is wettable without causing an allergic reaction when used as a treatment medium to treat a mammalian body.

One example of such a “biocompatible” treatment medium is a collagen, such as Type I collagen, in either dry powder form or semi-wet form that can be supersaturated with a selected antibiotic. Collagen is a group of naturally occurring proteins found in animals, especially in the flesh and connective tissues of mammals. It is the main component of connective tissue, and is the most abundant protein in mammals, making up about 25% to 35% of the whole-body protein content. Collagen, in the form of elongated fibrils, is mostly found in fibrous tissues such as tendon, ligament and skin, and is also abundant in cornea. Type I collagen is known in the relevant arts as a carrier for antibacterial drugs. See, for example, “Collagen as a Carrier For On-site Delivery of Antibacterial Drugs”, Advanced Drug Delivery Reviews, 55, (2003) 1679-1698.

Another example of a candidate biomaterial treatment medium is keratin, such as a keratin hydrogel in either powder form or semi-wet form that can be supersaturated with a selected antibiotic. Keratins are naturally derived fibrous structural proteins that can be fabricated into several biomaterial forms including hydrogels. See, “Keratin Hydrogels Support the Sustained Release of Bioactive Ciprofloxacin,” Society for Biomaterials, Jun. 16, 2011, Wiley Online Library, pages 544-553.

Another candidate biomaterial for use as the drug absorbing medium is chitosan. Chitosan is a linear polysaccharide which is produced commercially by deacetylation of chitin, which is the structural element in the exoskeleton of crustaceans (such as crabs and shrimp) and cell walls of fungi. If chitosan is used as the drug absorbing medium it can be in either dry powder form that can be congealed or in the form of a sponge that can be supersaturated with a selected antibiotic. See, “Chitosan Sponges to Locally Deliver Amikacin and Vancomycin”, Mar. 30, 2010, The Association of Bone and Joint Surgeons, Symposium Paper presented at the 2009 meeting of the Musculoskeletal Infection Society. Where the delivery medium is a sponge form, it will be necessary to cut, tear or un-peel the pouch, as described with respect to FIGS. 1-5.

The above biomaterial treatment mediums can be dispensed by squeezing the medium out the nozzle 49, except in the case of the chitosan sponge. Thus, as in the case of FIG. 1-5, a container pouch is provided formed of flexible plastic, the container pouch having an exterior, an interior and a sealed outer periphery, the sealed outer periphery forming a sterile interior region which isolates the pouch interior from a surrounding environment. In this case, the pouch is also provided with a dispensing nozzle 49, in addition to the syringe fitting 50. The drug absorbing medium, in this case a biomaterial, is placed within the pouch interior prior to sealing the outer periphery, whereby the sterile drug absorbing medium is initially isolated from the surrounding environment by the sealed periphery. The drug of choice would then be introduced within the interior of the pouch, as by discharging the drug through the syringe accepting fitting 50 provided on the pouch 47. After shaking or massaging the pouch to thoroughly mix the contents, the treatment medium can be discharged through the dispensing nozzle 49 onto a wound site.

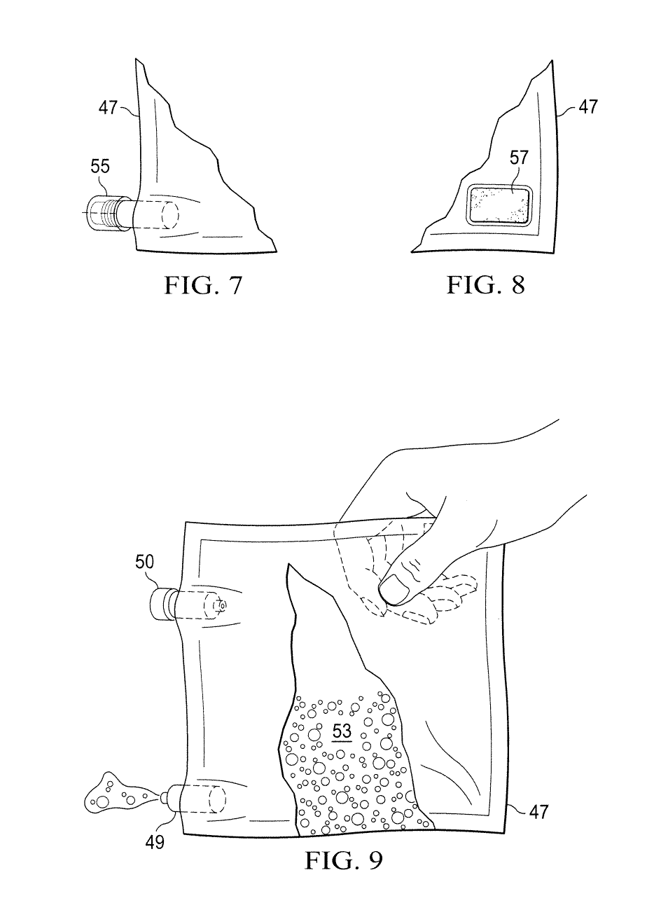

FIGS. 7 and 8 illustrate additional optional features which can be used with the pouch and delivery system of the invention. As shown in FIG. 7, the pouch 47 can be fitted with a screw-in butyl syringe adaptor 55 which can also be screwed off to accept a leuer lock fitting. In such a case, the adaptor tip could be cut off to allow the treatment medium to be squeezed out of the pouch. This would, in effect, combine the features of the separate syringe fitting and dispensing nozzle. As shown in FIG. 8, the pouch 47 can have a butyl patch 57 located strategically thereon to allow injection of an antibiotic drug using a larger than normal diameter needle, i.e., one not usable with the syringe accepting fitting 50.

An invention has been provided with several advantages. The sterile wound treatment kit of the invention is simple in design and economical to manufacture. The wound treatment kit can be assembled by making minor modifications to several commercially available treatment materials. The kits are shelf stable for an extended period of time and are only “activated” when a treatment drug of choice is injected through the syringe accepting fitting into the pouch interior. Applying the treatment medium locally has been found to be more effective than parenteral treatment methods in some cases providing a higher concentration of antibiotic locally at the wound site. For example, a typical through-the-vein antibiotic might have a volume of distribution of, at best, 1 gram per 5.6 liters of fluid. More commonly, these antibiotics penetrate into many areas of the body (e.g. brain, skin, lungs, etc.) and as a result, the concentration that is delivered to the site of injury is much less because of the antibiotics wide distribution. The reduced concentration at the injured site may not allow for antibiotic concentrations that are above the minimum inhibitory concentration require to inhibit the growth of the bacteria contaminating the wound. The above described wound treatment method would provide a greater inhibitory concentration of antibiotic at the wound site. Applicant's treatment method would therefore provide well above the minimum inhibitory concentration of antibiotic desired, with minimal risks of side effects.

While the invention has been shown in only two of its forms, it is thus limited but is susceptible to various changes and modifications without departing from the spirit thereof.

What is claimed is:

1. A sterile wound treatment kit for treating an open wound site, comprising:
a container pouch formed of flexible plastic, the container pouch having an exterior, an interior and a sealed outer periphery, the sealed outer periphery forming a sterile interior region which isolates the pouch interior from a surrounding environment;
a biomaterial drug absorbing medium located within the pouch interior and initially isolated from the surrounding environment by the sealed periphery;
a syringe accepting fitting located on the pouch for discharging a drug treatment from a syringe into the pouch interior;
a dispensing nozzle located on the pouch and communicating the interior and exterior thereof, whereby once a drug is injected into the sterile interior region and contacts and impregnates the drug absorbing medium, the drug absorbing medium can be discharged from the pouch interior onto a wound site.

2. The sterile wound treatment kit of claim 1, wherein the drug absorbing medium is Type I collagen in either dry powder form or semi-wet form that can be supersaturated with a selected antibiotic.

3. The sterile wound treatment kit of claim 1, wherein the drug absorbing medium is Chitosan in either dry powder that can be congealed or in the form of a sponge that can be supersaturated with a selected antibiotic.

4. The sterile wound treatment kit of claim 1, wherein the drug absorbing medium is Keratin Hydro gel in either powder form or semi-wet form that can be supersaturated with a selected antibiotic.

5. The sterile wound treatment kit of claim 1, wherein the pouch is fitted with a screw-in butyl syringe adaptor which can also be screwed off to accept a leuer lock fitting.

6. The sterile wound treatment kit of claim 1, wherein the pouch is supplied with a butyl patch to allow injection of an antibiotic drug using a larger than normal diameter needle.

7. The sterile wound treatment kit of claim 1, wherein the treatment drug is a broad spectrum antibiotic.

8. The sterile wound treatment kit of claim 1, wherein the syringe accepting fitting is a Leur Lock fitting or other suitable syringe accepting fitting.

9. The sterile wound treatment kit of claim 1, wherein the container pouch is formed of two overlaying sheets of flexible plastic, the sheets of flexible plastic having an adhesive applied around an outer periphery thereof to form a sealed pouch and wherein the flexible plastic used to form the container pouch has a construction where the two plastic sides may be peeled apart from one another, or a thickness which is selected to allow the pouch to be opened by tearing or cutting.

10. A method of treating a wound by applying a sterile drug treatment medium, the method comprising the steps of:
providing a container pouch formed of flexible plastic, the container pouch having an exterior, an interior and a sealed outer periphery, the sealed outer periphery forming a sterile interior region which isolates the pouch interior from a surrounding environment;
placing a sterile drug absorbing medium within the pouch interior prior to sealing the outer periphery, whereby the sterile drug absorbing medium is initially isolated from the surrounding environment by the sealed periphery;
providing a syringe accepting fitting on the pouch for discharging a drug treatment from a syringe into the pouch interior;
providing a dispensing nozzle on the pouch which communicates the interior and exterior thereof, whereby once a drug is injected into the sterile interior region and contacts and impregnates the drug absorbing medium, the drug absorbing medium can be discharged from the pouch interior through the dispensing nozzle onto a wound site;
installing a syringe within the fitting on the container pouch and discharging a selected treatment drug from the syringe into the pouch interior;
allowing the drug absorbing medium to contact and evenly absorb become impregnated with the treatment drug;
opening the dispensing nozzle; and
squeezing the pouch to cause the impregnated medium to be dispensed through the nozzle onto a wound site.

11. The method of claim 10, wherein the container pouch is opened by cutting the pouch with scissors.

12. The method of claim 10, wherein the container pouch is opened by tearing the pouch.

13. The method of claim 10, wherein the container pouch containing drug absorbing medium is supplied in bulk to a medical facility and wherein the treatment drug is injected into the pouch interior region shortly before application to the wound of a patient.
